(19) United States
(12) Patent Application Publication
(10) Pub. No.: US 2026/0268482 A1
(43) Pub. Date: Sep. 10, 2026

(54) FINE-TUNING MEDICAL IMAGE FOUNDATIONAL MODELS WITH CLINCIAL EXPERTS IN THE LOOP

(71) Applicant: Siemens Healthineers AG, Forchheim (DE)

(72) Inventors: Yue Zhang, Short Hills, NJ (US); Puneet Sharma, Princeton Junction, NJ (US)

(21) Appl. No.: 19/072,357

(22) Filed: Mar. 6, 2025

(51) Int. Cl. G06T 7/00; G16H 80/00

(52) U.S. Cl. CPC G06T 7/0012; G16H 80/00; G06T 2207/20076; G06T 2207/20084; G06T 2207/30168

(57) ABSTRACT

Systems and methods for fine-tuning a pretrained medical foundational model are provided. An uncertainty associated with weakly labeled training medical images is determined. Unconfident ones of the weakly labeled training medical images are selected based on the uncertainty. Labels for the selected unconfident weakly labeled training medical images are received from one or more users. A machine learning based student network is fine-tuned based on the received labels and the selected unconfident weakly labeled training medical images using a machine learning based teacher network. The machine learning based teacher network and the machine learning based student network are initialized as being a pretrained medical foundational model during a first iteration. The machine learning based teacher network is updated based on weights of the fine-tuned machine learning based student network and the determining, the selecting, the receiving, and the fine-tuning are repeated.

### TECHNICAL FIELD

The present invention relates generally to medical foundational models, and in particular to fine-tuning medical image foundational models with clinical experts in the loop.

### BACKGROUND

Foundational models are a type of AI (artificial intelligence) models pretrained on a vast dataset to serve as a broad, adaptable base for various applications. In the medical domain, there are various types of medical foundational models, including, for example, modality-specific, organ-specific, and task-specific medical foundational models, depending on the medical application. Prior to applying pretrained foundational models to unseen domains, pretrained foundational models are typically fine-tuned. Conventionally, pretrained foundational models are fine-tuned to the medical domain using large amounts of annotated medical images. However, the annotation of such large amounts of medical images is a time-consuming and costly process.

### BRIEF SUMMARY OF THE INVENTION

In accordance with one or more embodiments, systems and methods for performing a medical imaging analysis task using a fine-tuned medical foundational model are provided. One or more input medical images are received. A medical imaging analysis task is performed based on the one or more input medical images using a fine-tuned medical foundational model. Results of the medical imaging analysis task are output. The medical foundational model is fine-tuned by receiving weakly labeled training medical images. An uncertainty associated with each of the weakly labeled training medical images is determined. Unconfident ones of the weakly labeled training medical images are selected based on the uncertainty. Labels for the selected unconfident weakly labeled training medical images are received from one or more users. A machine learning based student network is fine-tuned based on the received labels and the selected unconfident weakly labeled training medical images using a machine learning based teacher network. The machine learning based teacher network and the machine learning based student network are initialized as being a pretrained medical foundational model during a first iteration. The machine learning based teacher network is updated based on weights of the fine-tuned machine learning based student network and the determining, the selecting, the receiving, and the fine-tuning are repeated for one or more additional iterations. The machine learning based student network is initialized as being the updated machine learning based teacher network during the one or more additional iterations. The trained machine learning based student network is output as the fine-tuned medical foundational model.

In one embodiment, for each respective one of the weakly labeled training medical images, a plurality of perturbations is applied to the respective weakly labeled training medical image. A prediction is generated for each of the plurality of perturbations using the machine learning based teacher network. The uncertainty of the respective weakly labeled training medical image is determined based on the predictions.

In one embodiment, selecting unconfident ones of the weakly labeled training medical images based on the uncertainty comprises comparing the uncertainty to a threshold.

In one embodiment, selecting unconfident ones of the weakly labeled training medical images comprises sampling the unconfident ones of the weakly labeled training medical images within ranges of the uncertainty.

In one embodiment, selecting unconfident ones of the weakly labeled training medical images comprises determining confident ones of the weakly labeled training medical images based on the uncertainty and fine-tuning a machine learning based student network comprises fine-tuning the machine learning based student network further based on the confident weakly labeled training medical images and their weak labels.

In one embodiment, updating the machine learning based teacher network comprises updating weights of the machine learning based teacher network based on an exponential moving average of weights of the fine-tuned machine learning based student network.

In one embodiment, fine-tuning a machine learning based student network comprises fine-tuning the machine learning based student network based on a consistency loss enforcing a consistency between outputs of the machine learning based teacher network and the machine learning based student network and a supervision loss measuring an error between the outputs of the machine learning based student network and the received labels.

In accordance with one or more embodiments, systems and methods for fine-tuning a pretrained medical foundational model are provided. Weakly labeled training medical images are received. An uncertainty associated with each of the weakly labeled training medical images is determined. Unconfident ones of the weakly labeled training medical images are selected based on the uncertainty. Labels for the selected unconfident weakly labeled training medical images are received from one or more users. A machine learning based student network is fine-tuned based on the received labels and the selected unconfident weakly labeled training medical images using the machine learning based teacher network. The machine learning based teacher network and the machine learning based student network are initialized as being the pretrained medical foundational model during a first iteration. The machine learning based teacher network is updated based on weights of the fine-tuned machine learning based student network and the determining, the selecting, the receiving, and the fine-tuning are repeated for one or more additional iterations. The machine learning based student network is initialized as being the updated machine learning based teacher network during the one or more additional iterations. The trained machine learning based student network is output as a fine-tuned medical foundational model.

These and other advantages of the invention will be apparent to those of ordinary skill in the art by reference to the following detailed description and the accompanying drawings.

### BRIEF DESCRIPTION OF THE DRAWINGS

FIG. 1 shows a workflow for fine-tuning a pretrained medical foundational model, in accordance with one or more embodiments;

FIG. 2 shows a method for fine-tuning a medical foundational model, in accordance with one or more embodiments;

FIG. 3 shows a workflow for fine-tuning a medical foundational model, in accordance with one or more embodiments;

FIG. 4 shows images comparing strong (accurate) labels with weak labels, in accordance with one or more embodiments;

FIG. 5 shows a flowchart illustrating benefits of sampling weakly labeled training medical images for annotation, in accordance with one or more embodiments;

FIG. 6 shows a method for performing a medical imaging analysis task using a fine-tuned medical foundational model, in accordance with one or more embodiments;

FIG. 7 shows an exemplary artificial neural network that may be used to implement one or more embodiments;

FIG. 8 shows a convolutional neural network that may be used to implement one or more embodiments;

FIG. 9 shows a schematic structure of a recurrent machine learning model that may be used to implement one or more embodiments; and

FIG. 10 shows a high-level block diagram of a computer that may be used to implement one or more embodiments.

### DETAILED DESCRIPTION

The present invention generally relates to methods and systems for fine-tuning medical foundational models with clinical experts in the loop. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for the fine-tuning of medical foundational models with reduced amounts of annotated training medical images. The fine-tuning of medical foundational models in accordance with embodiments described herein applies a sample selection scheme to automatically select a representative subset of medical images for annotation by a clinician for fine-tuning the medical foundational model. Advantageously, the fine-tuning of medical foundational models in accordance with embodiments described herein is performed with reduced amounts of annotated medical images as compared with conventional approaches, while also improving model performance by using both annotated and unannotated medical images.

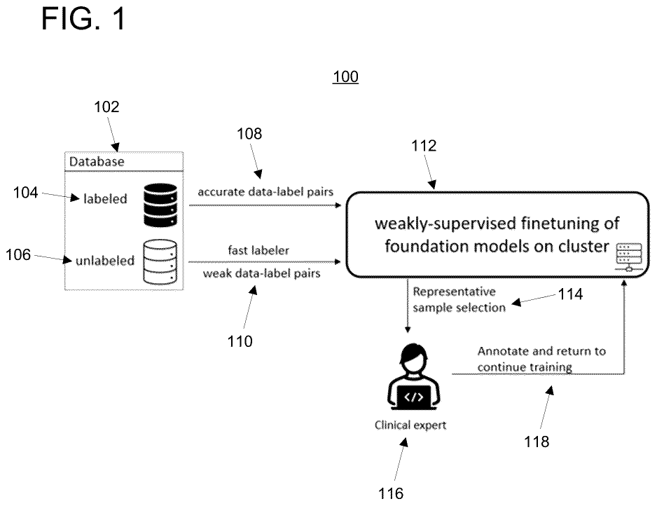

FIG. 1 shows a workflow 100 for fine-tuning a pretrained medical foundational model, in accordance with one or more embodiments. The pretrained medical foundational model is pretrained on a vast dataset during a prior stage. The pretrained medical foundational model is then fine-tuned using a database 102 comprising labeled (or annotated) training medical images 104 and unlabeled (or unannotated) training medical images 106. The labeled training medical images 104 represent accurate data-label pairs 108 that can be directly utilized in the fine-tuning of the medical foundational model. For the unlabeled training medical images 106, a fast labeler is applied to generate weak labels, thereby forming weak data-label pairs 110.

Accurate data-label pairs 108 and weak data-label pairs 110 are employed within a framework 112 for weakly-supervised fine-tuning of the medical foundational model using student-teacher training. Typically, unlabeled training medical images 106 comprise both easy and difficult cases. The classification of easy and difficult cases is contingent upon the efficacy of the medical foundational model. The medical foundational model can proficiently handle the easy cases, while encountering difficulty with the difficult cases. Recognizing and labeling the difficult cases in unlabeled training medical images 106 can significantly enhance performance of the fine-tuned medical foundational model, particularly in scenarios where annotation resources are scarce.

A representative sample selection 114 is performed to autonomously distinguish between easy and difficult cases in unlabeled training medical images 106, without relying on ground truth accurate labels. Cases identified as being difficult are forwarded to a clinical expert 116 (or any other user) for annotation 118. Framework 112 is performed for fine-tuning the medical foundational model according to student-teacher training using the labeled difficult cases and the weakly labeled easy cases of weak data-label pairs 110, along with accurate data-label pairs 108.

In framework 112, student-teacher training is applied to iteratively transfer the knowledge of a machine learning based teacher network to a machine learning based student network. At the first iteration, the machine learning based teacher network is initialized as being the pretrained medical foundational model and, at each iteration, the machine learning based student network is initialized as being a copy of the machine learning based teacher network. The machine learning based student network is updated using the labeled difficult cases and the weakly labeled easy cases of weak data-label pairs 110, along with accurate data-label pairs 108. Subsequently, the machine learning based teacher model is updated based on the updated machine learning based student network and the updated machine learning based teacher network is utilized to re-identify the difficult cases in unlabeled training medical images 106, thus iterating the training process. The output of framework 112 is the trained machine learning based student network, which serves as the final fine-tuned foundational model.

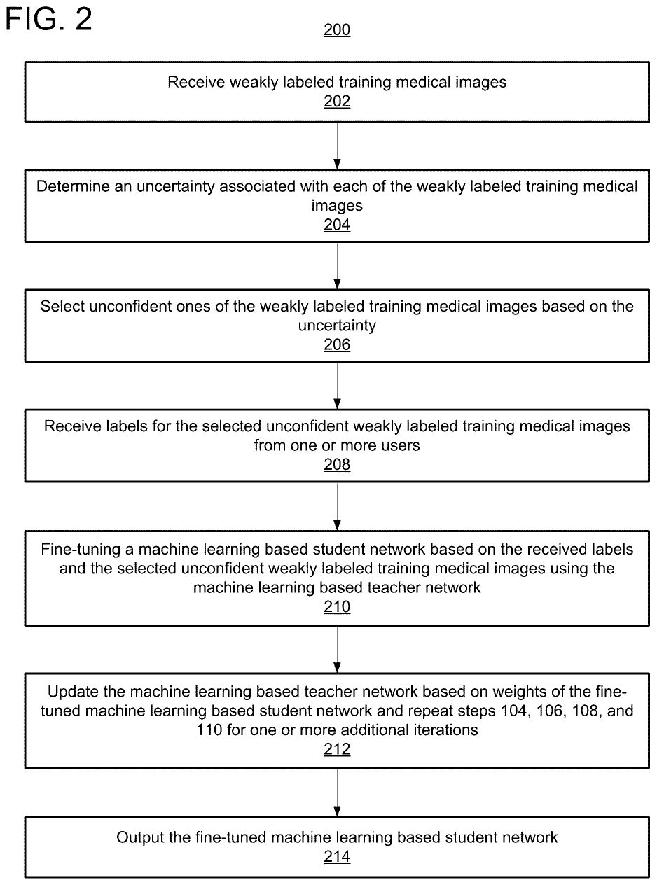

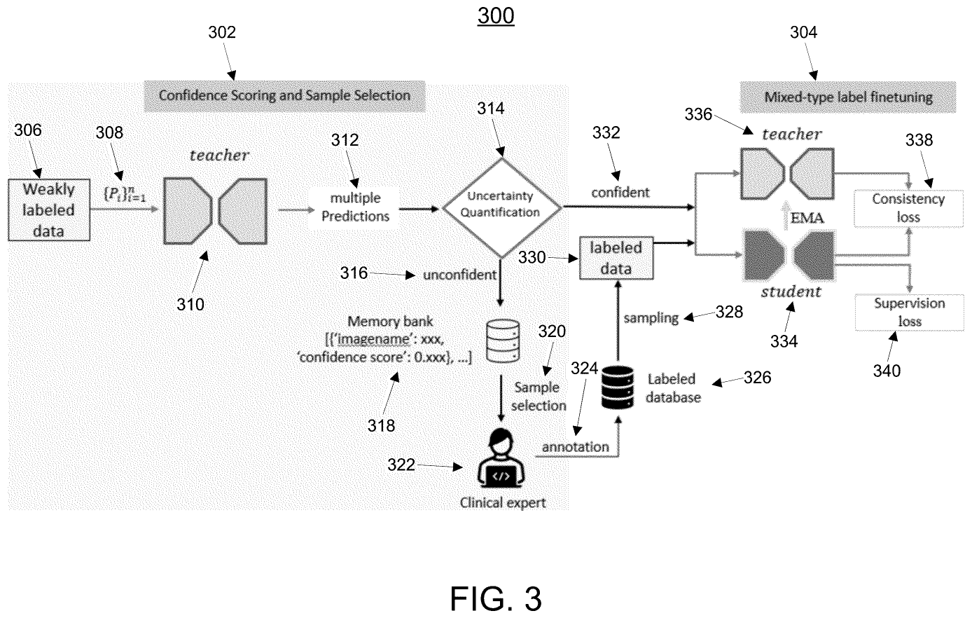

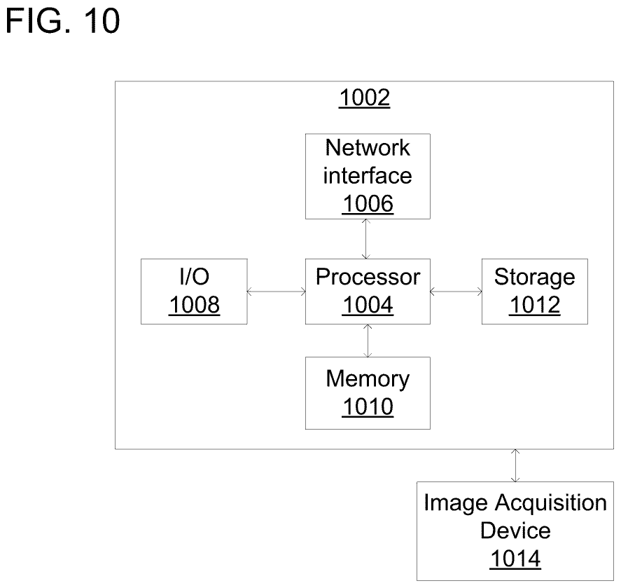

FIG. 2 shows a method 200 for fine-tuning a medical foundational model, in accordance with one or more embodiments. The steps and sub-steps of method 200 may be performed by one or more suitable computing devices, such as, e.g., computer 1002 of FIG. 10. FIG. 3 shows a workflow 300 for fine-tuning a medical foundational model, in accordance with one or more embodiments. Workflow 300 comprises a confidence scoring and sample selection stage 302 and a mixed-type label fine-tuning stage 304. FIG. 2 and FIG. 3 will be described together. The steps of method 200 of FIG. 2 and workflow 300 of FIG. 3 are performed during a training stage for fine-tuning the medical foundational model.

At step 202 of FIG. 2, weakly labeled training medical images are received. In one example, as shown in workflow 300 of FIG. 3, the weakly labeled training medical images are weakly labeled data 306.

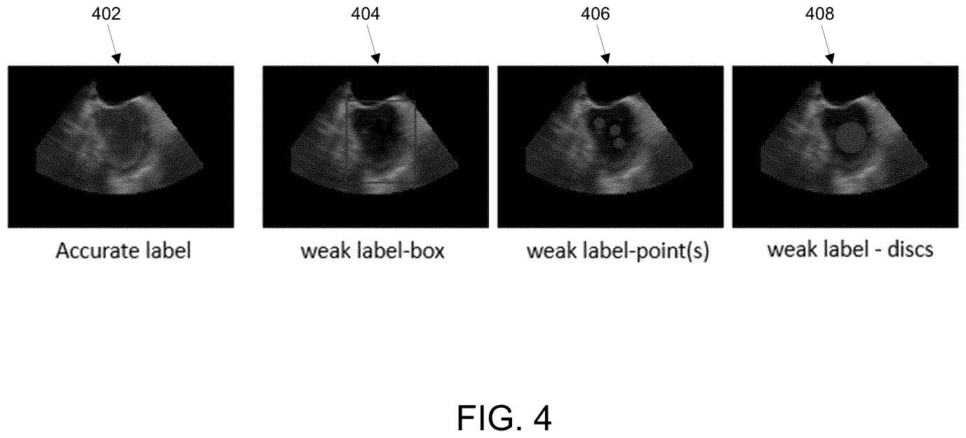

The weakly labeled training medical images comprises training medical images and corresponding weak labels. Weak labels refer to approximate labels or annotations that are typically less precise, less reliable, or less detailed than strong or high-quality labels (e.g., ground truth labels). The weak labels may be represented as boxes, points, discs, or in any other suitable form. FIG. 4 shows images 400 comparing strong (accurate) labels with weak labels, in accordance with one or more embodiments. Image 402 depicts a manually annotated ground truth accurate label, image 404 shows a weak label box, image 406 shows weak label points, and image 408 show weak label discs. The weak labels are generated by a fast labeler. In one embodiment, the fast labeler is a machine learning based network trained to generate the weak labels. Since such a machine learning based network is not required to provide dense pixel-level or highly precise labels, the machine learning based network does not require a large quantity of precisely annotated training data. In another embodiment, the fast labeler is a human annotator. Generating weak labels is significantly quicker and simpler than performing pixel-wise annotations for human annotators.

Returning to step 202 of FIG. 2, the weakly labeled training medical images may depict an anatomical object of a patient, such as, e.g., organs, bones, vessels, tumors or abnormalities, or any other anatomical object or objects of interest. The weakly labeled training medical images may be of any suitable modality, such as, e.g., MRI (magnetic resonance imaging), CT (computed tomography), US (ultrasound), x-ray, or any other medical imaging modality or combinations of medical imaging modalities. The weakly labeled training medical images may comprise 2D (two dimensional) images and/or 3D (three dimensional) volumes, and may comprise any number of medical images.

The weakly labeled training medical images may be received, for example, by directly receiving the images from an image acquisition device (e.g., image acquisition device 1014 of FIG. 10) as the weakly labeled training medical images are acquired, by loading the weakly labeled training medical images from a storage or memory of a computer system (e.g., storage 1012 or memory 1010 of computer 1002 of FIG. 10), or by receiving the weakly labeled training medical images from a remote computer system (e.g., computer 1002 of FIG. 10). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 204 of FIG. 2, an uncertainty associated with each of the weakly labeled training medical images is determined. The uncertainty associated with each of the weakly labeled training medical images is determined using a machine learning based teacher network. The objective is to determine whether each weakly labeled training medical image is considered an “easy” case of a “difficult” case.

In one embodiment, with continued reference to workflow 300 of FIG. 3, the uncertainty is determined by applying perturbations 308 to the weakly labeled data 306. The assumption is that machine learning based teacher network 310 will perform consistently on the easy cases even when perturbations 308 are applied, while perform inconsistently on the difficult cases when perturbations 308 are applied. Given an image x_{w} and its weak label y_{w}, perturbed pairs {Pᵢ(x_{w})},{Pᵢ(y_{w})} are generated, where i=1, 2, . . . , n and Pᵢ represents a class of perturbation functions. The perturbation functions may include, for example, noise-based perturbations (e.g., Gaussian noise, etc.), geometric transformations (e.g., random rotation, translation, scaling, etc.), color/intensity changes (e.g., brightness, contrast, etc.), or any other suitable perturbation functions. The machine learning based teacher network 310 is denoted f^{t}. For each image x_{w}, multiple predictions {f^{t}(Pᵢ(x_{w}))}, i=1, 2, . . . , n 312 are generated using teacher network f^{t} 310 based on the perturbations 308.

An uncertainty quantification module [symbol] 314 is then leveraged to identify the easiness (or difficulty) of the weak labels. Given a list of the multiple predictions {f^{t}(Pᵢ(x_{w}))} 310, uncertainty quantification module [symbol] 314, maps the list to a single scalar confidence score sϵ[0,1] reflecting the consistency between the multiple predictions {f^{t}(Pᵢ(x_{w}))} 310. The uncertainty quantification module [symbol] 314 may be implemented using any suitable approach. For example, the uncertainty quantification module [symbol] 314 may be implemented using a deterministic function (e.g., standard deviation) or a learned-based approach (e.g., machine learning based network). The confidence score s represents the uncertainty associated with the weak labels.

At step 206 of FIG. 2, unconfident ones of the weakly labeled training medical images are selected based on the uncertainty. With continued reference to workflow 300 of FIG. 3, given a predefined confidence threshold, the weak labels may be classified to be “confident” 332 or “unconfident” 316 by comparing the confidence score s with the confidence threshold.

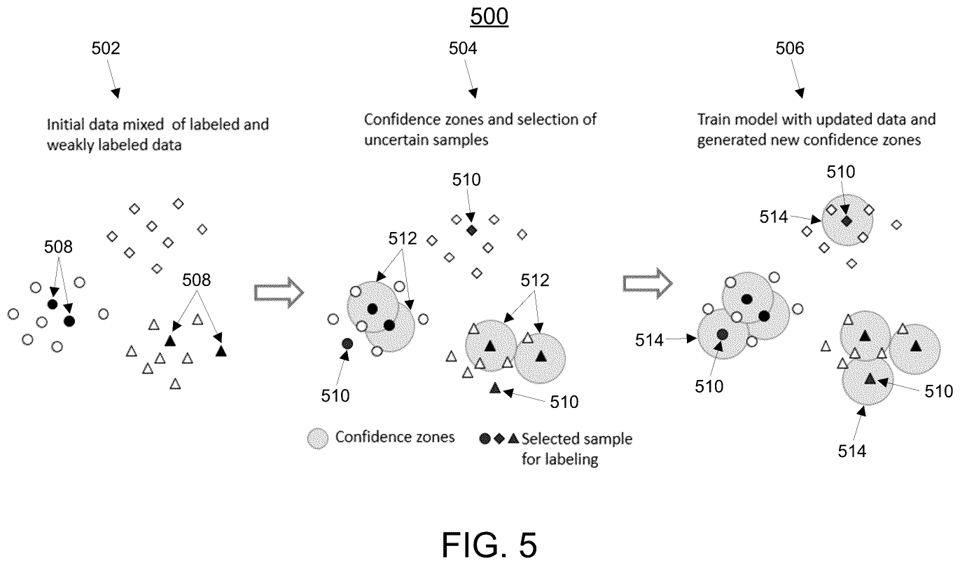

For weak labels classified as being unconfident, the weakly labeled training medical images and their confidence scores s are stored in a database or memory bank 318. Memory bank 318 stores all of the weakly labeled training medical images on which the machine learning based teacher network performs inconsistently (i.e., having unconfident weak labels). In one embodiment, all of the weakly labeled training medical images having weak labels classified as being unconfident (i.e., all weakly labeled training medical images stored in memory bank 318) are selected as being the unconfident weakly labeled training medical images. Annotating weakly labeled training medical images in memory bank 318 will bring better guidance and model improvement when fine-tuning the medical foundational model. However, if the confidence threshold is defined to be too high, many weakly labeled training medical images will be classified as being unconfident and thus memory bank 318 can be big. Annotating all weakly labeled training medical images in memory bank 318 can thus be time consuming. To mitigate this, in another embodiment, the weakly labeled training medical images in memory bank 318 may be selected 320 by uniformly sampling the weakly labeled training medical images at different ranges of the confidence scores. For example, N weakly labeled training medical images may be sampled from confidence scores within each range [0,0.1], [0.1,0.2], etc. FIG. 5 shows benefits of sampling weakly labeled training medical images for annotation, described in detail below.

At step 208 of FIG. 2, labels for the selected unconfident weakly labeled training medical images are received from one or more users. For example, as shown in workflow 300 of FIG. 3, the selected unconfident weakly labeled training medical images are forwarded to clinical expert 322, who generates annotations 324. Annotations 324 are stored in labeled database 326. Annotations 324 are ground truth annotations that are more precise, reliable, and/or detailed as compared with the weak labels.

At step 210 of FIG. 2, a machine learning based student network is fine-tuned based on the received labeled and the selected unconfident weakly labeled training medical images using the machine learning based teacher network. The machine learning based teacher network and the machine learning based student network are initialized as being the pretrained foundational model during the first iteration. In one example, as shown in workflow 300 of FIG. 3, the labeled database 326 are sampled 328 to identify labeled data 330 for student-teacher training. The sampling 328 can be deterministic (e.g., uniform sampling) or data-driven (e.g., weighted sampling based on the corresponding confidence scoring in memory bank 318, where the lower the confidence score the higher the probability the data will be sampled 328).

In student-teacher training, the machine learning based teacher network f^{t} 336 is typically the heavier network that is highly accurate but slow and/or resource intensive while the machine learning based student network f^{s} 334 is typically the lightweight network with fewer parameters. The output of the machine learning based teacher network f^{t} 336 is used to iteratively guide the machine learning based student network f^{s} 334. It should be understood that while machine learning based teacher networks 310 and 336 are separately shown to facilitate illustration of workflow 300, machine learning based teacher networks 310 and 336 are the same machine learning based teacher network.

At the beginning of the first iteration, the machine learning based teacher network f^{t} 336 is initialized as being a copy of the pretrained medical foundational model and the machine learning based student network f^{s} 334 is initialized as being a copy of machine learning based teacher network f^{t} 336. During the first iteration, the weights of the machine learning based teacher network f^{t} 336 are frozen while the weights of the machine learning based student network f^{s} 334 are updated. At the beginning of each subsequent iteration, the weights of the machine learning based teacher network f^{t} 336 are then updated based on the weights of the updated machine learning based student network f^{s} 334 (as updated during the prior iteration) and the machine learning based student network f^{s} 334 is initialized as being a copy of updated machine learning based teacher network f^{t} 336. The weights of the machine learning based teacher network f^{t} 336 are frozen while the weights of the machine learning based student network f^{s} 334 are updated. The machine learning based student network f^{s} 334 is iteratively updated, thereby representing the fine-tuned medical foundational model.

The machine learning based student network f^{s} 334 is fine-tuned using weakly labeled training medical images labeled as being confident 332 as well as the weakly labeled training medical images that were annotated as labeled data 330. The machine learning based student network f^{s} 334 is fine-tuned using a combination loss function of consistency loss L_{consistency} 338 enforcing a consistency between outputs of the machine learning based teacher network f^{t} 336 and the machine learning based student network f^{s} 334 and supervision loss Lₛᵤₚₑᵣᵥᵢₛᵢₒₙ 340 measuring an error between outputs of the machine learning based student network f^{s} 334 and the labels, according to Equation (1):

$$L = {L}_{c o n s i s t e n c y} ( H ( {f}^{t} ( {x}_{u} ) ) , {f}^{s} ( {x}_{u} ) ) + {L}_{supervision} ( {f}^{s} ( {x}_{l} ) , {y}_{l} ) \qquad ( 1 )$$

where H is a hard thresholding function which binarizes the prediction to be 0 or 1. Consistency loss L_{consistency} 338 and supervision loss Lₛᵤₚₑᵣᵥᵢₛᵢₒₙ 340 can be a task-dependent loss function, such as, e.g., cross entropy for classification or soft dice score for image segmentation. During each iteration, the weights θ^{t} of the machine learning based teacher network f^{t} 336 are frozen while weights θ^{s} of the machine learning based student network f^{s} 334 are updated according to the loss function of Equation (1).

At step 212 of FIG. 2, the machine learning based teacher network is updated based on weights of the fine-tuned machine learning based student network and the determining (step 204), the selecting (step 206), the receiving (step 208), and the fine-tuning (step 210) are repeated for one or more additional iterations. The machine learning based student network is initialized as being the updated machine learning based teacher network during the one or more additional iterations.

In one embodiment, the weights θ^{t} of the machine learning based teacher network f^{t} 336 may be updated as the EMA (exponential moving average) of the weights θ^{s} of the machine learning based student network f^{s} 334 as in Equation (2):

$${θ}_{i t}^{t} = {αθ}_{i t - 1}^{t} + ( 1 - α ) {θ}_{i t}^{s} \qquad ( 2 )$$

where the subscript it denotes the training step and α is a scalar number that controls the weight balance between the weights θ^{t} of teacher network f^{t} 336 from the previous iteration and the weights θ^{s} of student network f^{s} 334 from the current iteration. In one embodiment, α is set to 0.99.

The repeating may be performed for the one or more additional iterations until a stopping condition is satisfied (e.g., convergence of the loss function, accuracy plateau, error threshold, a predetermined number of iterations, etc.). Upon completion of the one or more additional iterations, the trained machine learning based student network serves as the final fine-tuned foundational model.

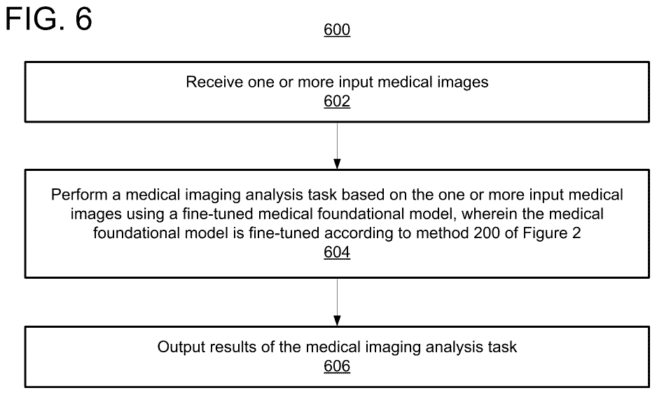

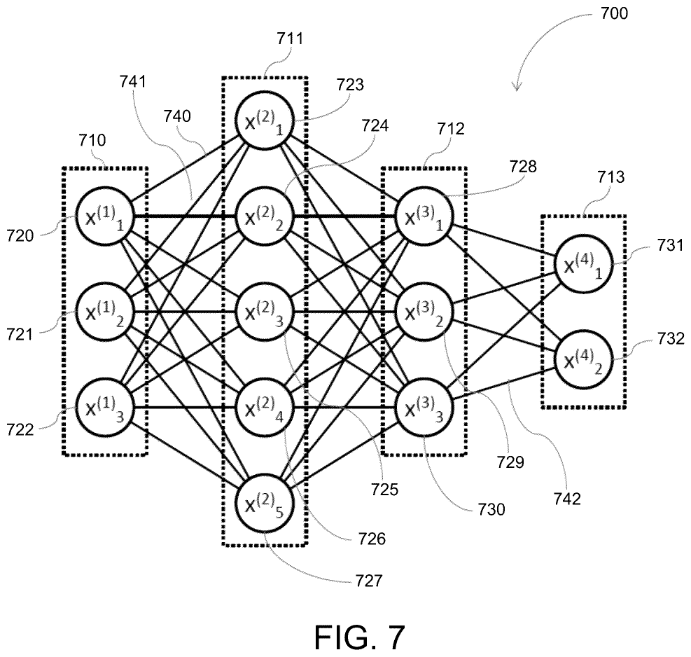

At step 214 of FIG. 2, the trained machine learning based student network is output. For example, the trained machine learning based student network can be output by storing the trained machine learning based student network on a memory or storage of a computer system (e.g., memory 1010 or storage 1012 of computer 1002 of FIG. 7) or by transmitting the trained machine learning based student network to a remote computer system (e.g., computer 1002 of FIG. 10). In one embodiment, the trained machine learning based student network is applied during an inference stage to perform a medical imaging analysis task, e.g., according to method 600 of FIG. 6.

FIG. 5 shows a flowchart 500 illustrating benefits of sampling weakly labeled training medical images for annotation, in accordance with one or more embodiments. In flowchart 500, different shapes represent different classes (e.g., diseases) of data. Step 502 shows an initial data mix for training a medical foundational model. In step 502, the filled shapes represent labeled data 508 and the unfilled shapes (i.e., the remaining shapes) represent weakly labeled data. Step 504 shows confidence zones 512 of the medical foundational model when fine-tuned on the mix of data shown in step 502. Confidence zones 512 represent data zones of high confidence of the medical foundational model when fine-tuned on the mix of data shown in step 502. As the medical foundational model is trained to excel on data resembling the labeled data 508, the medical foundational model is fine-tuned to have confidence zones 512 around labeled data 508. Conversely, the medical foundational model may struggle with data dissimilar to the labeled data. This is illustrated in step 504, where there is no labeled data, and thus no confidence zones, for the diamond class of data. A sample selection is thus performed to select unlabeled data 510 for labeling, thus resulting in unlabeled data 510 being labeled data. At step 506, the medical foundational model is fine-tuned with the mix of data shown in step 504 (with labels for the data 510), thus developing confidence zones 514 around the data 510. Steps 504 and 506 may be iteratively repeated to fine-tune the medical foundational model to develop confidence zones around all data.

FIG. 6 shows a method 600 for performing a medical imaging analysis task using a fine-tuned medical foundational model, in accordance with one or more embodiments. The steps and sub-steps of method 600 may be performed by one or more suitable computing devices, such as, e.g., computer 1002 of FIG. 10. The steps of method 600 of FIG. 6 are performed during an inference stage for applying the fine-tuned medical foundational model.

At step 602 of FIG. 6, one or more input medical images are received. The one or more input medical images may depict any anatomical object of interest and may be of any suitable modality. The one or more input medical images may comprise 2D images and/or 3D volumes. The one or more input medical images may be received, for example, by directly receiving the images from an image acquisition device (e.g., image acquisition device 1014 of FIG. 10) as the one or more input medical images are acquired, by loading the one or more input medical images from a storage or memory of a computer system (e.g., storage 1012 or memory 1010 of computer 1002 of FIG. 10), or by receiving the one or more input medical images from a remote computer system (e.g., computer 1002 of FIG. 10).

At step 604 of FIG. 6, a medical imaging analysis task is performed based on the one or more input medical images using a fine-tuned medical foundational model. The medical foundational model is fine-tuned according to method 200 of FIG. 2 and/or workflow 300 of FIG. 3. The medical imaging analysis task may comprise, for example, segmentation, registration, classification, detection, or any other medical imaging analysis task or tasks. The medical foundational model receives as input the one or more input medical images and generates as output results of the medical imaging analysis task.

At step 606 of FIG. 6, results of the medical imaging analysis task are output. For example, the results of the medical imaging analysis task can be output by displaying the results on a display device of a computer system (e.g., I/O 1008 of computer 1002 of FIG. 10), storing the results on a memory or storage of a computer system (e.g., memory 1010 or storage 1012 of computer 1002 of FIG. 10), or by transmitting the results to a remote computer system (e.g., computer 1002 of FIG. 10).

Advantageously, embodiments described herein leverage both semi-supervised learning and active learning for fine-tuning the medical foundational model. Compared to fully supervised fine-tuning, embodiments described herein do not require such large amounts of labeled training data. With active learning, only a subset of representative samples will be selected for annotation, greatly reducing the annotation costs. This is especially important in the medical domain where clinical experts' time is expensive. Compared to active learning only fine-tuning, embodiments described herein also utilizes unlabeled data. While active learning can select a set of representative samples, fine-tuning a medical foundational model on this small set can cause overfitting. The semi-supervised fine-tuning in accordance with embodiments described herein mitigates this risk and improves model robustness and generalizability.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the medical foundational model fine-tuned according to framework 112 of FIG. 1, the machine learning based teacher network, the machine learning based student network, and the medical foundational model utilized in method 200 of FIG. 2, the machine learning based teacher network 310 and 336 and the machine learning based student network 334 in workflow 300 of FIG. 3, and the medical foundational model utilized at step 604 of FIG. 6, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

FIG. 7 shows an embodiment of an artificial neural network 700 that may be used to implement one or more machine learning models described herein. Alternative terms for “artificial neural network” are “neural network”, “artificial neural net” or “neural net”.

The artificial neural network 700 comprises nodes 720, . . . , 732 and edges 740, . . . 742, wherein each edge 740, . . . , 742 is a directed connection from a first node 720, . . . , 732 to a second node 720, . . . , 732. In general, the first node 720, . . . , 732 and the second node 720, . . . , 732 are different nodes 720, . . . , 732, it is also possible that the first node 720, . . . , 732 and the second node 720, . . . , 732 are identical. For example, in FIG. 7 the edge 740 is a directed connection from the node 720 to the node 723, and the edge 742 is a directed connection from the node 730 to the node 732. An edge 740, . . . , 742 from a first node 720, . . . , 732 to a second node 720, . . . , 732 is also denoted as “ingoing edge” for the second node 720, . . . , 732 and as “outgoing edge” for the first node 720, . . . , 732.

In this embodiment, the nodes 720, . . . , 732 of the artificial neural network 700 can be arranged in layers 710, . . . , 713, wherein the layers can comprise an intrinsic order introduced by the edges 740, . . . 742 between the nodes 720, . . . , 732. In particular, edges 740, . . . , 742 can exist only between neighboring layers of nodes. In the displayed embodiment, there is an input layer 710 comprising only nodes 720, . . . , 722 without an incoming edge, an output layer 713 comprising only nodes 731, 732 without outgoing edges, and hidden layers 711, 712 in-between the input layer 710 and the output layer 713. In general, the number of hidden layers 711, 712 can be chosen arbitrarily. The number of nodes 720, . . . , 722 within the input layer 710 usually relates to the number of input values of the neural network, and the number of nodes 731, 732 within the output layer 713 usually relates to the number of output values of the neural network.

In particular, a (real) number can be assigned as a value to every node 720, . . . , 732 of the neural network 700. Here, x⁽ⁿ⁾ᵢ denotes the value of the i-th node 720, . . . , 732 of the n-th layer 710, . . . , 713. The values of the nodes 720, . . . , 722 of the input layer 710 are equivalent to the input values of the neural network 700, the values of the nodes 731, 732 of the output layer 713 are equivalent to the output value of the neural network 700. Furthermore, each edge 740, . . . , 742 can comprise a weight being a real number, in particular, the weight is a real number within the interval [−1, 1] or within the interval [0, 1]. Here, w^{(m,n)}_{i,j} denotes the weight of the edge between the i-th node 720, . . . , 732 of the m-th layer 710, . . . , 713 and the j-th node 720, . . . , 732 of the n-th layer 710, . . . , 713. Furthermore, the abbreviation w⁽ⁿ⁾_{i,j} is defined for the weight w^{(n,n+1)}_{i,j}.

In particular, to calculate the output values of the neural network 700, the input values are propagated through the neural network. In particular, the values of the nodes 720, . . . , 732 of the (n+1)-th layer 710, . . . , 713 can be calculated based on the values of the nodes 720, . . . , 732 of the n-th layer 710, . . . , 713 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 710 are given by the input of the neural network 700, wherein values of the first hid-den layer 711 can be calculated based on the values of the input layer 710 of the neural network, wherein values of the second hidden layer 712 can be calculated based in the values of the first hidden layer 711, etc.

In order to set the values w^{(m,n)}_{i,j} for the edges, the neural network 700 has to be trained using training data. In particular, training data comprises training input data and training output data (denoted as tᵢ). For a training step, the neural network 700 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 700 (backpropagation algorithm). In particular, the weights are changed according to

$${{w}^{′}}^{{( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 713, wherein f′ is the first derivative of the activation function, and t⁽ⁿ⁺¹⁾; is the comparison training value for the j-th node of the output layer 713.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

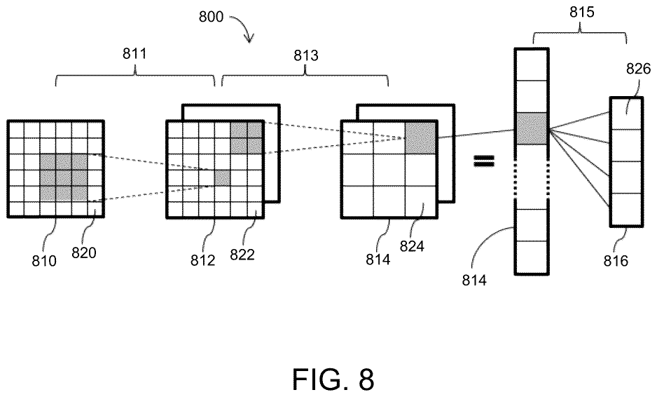

FIG. 8 shows an embodiment of a convolutional neural network 800 that may be used to implement one or more machine learning models described herein. In the displayed embodiment, the convolutional neural network 800 comprises an input node layer 810, a convolutional layer 811, a pooling layer 813, a fully connected layer 814 and an output node layer 816, as well as hidden node layers 812, 814. Alternatively, the convolutional neural network 800 can comprise several convolutional layers 811, several pooling layers 813 and several fully connected layers 815, as well as other types of layers. The order of the layers can be chosen arbitrarily, usually fully connected layers 815 are used as the last layers before the output layer 816.

In particular, within a convolutional neural network 800 nodes 820, 822, 824 of a node layer 810, 812, 814 can be considered to be arranged as a d-dimensional matrix or as a d-dimensional image. In particular, in the two-dimensional case the value of the node 820, 822, 824 indexed with i and j in the n-th node layer 810, 812, 814 can be denoted as x(n)[i, j]. However, the arrangement of the nodes 820, 822, 824 of one node layer 810, 812, 814 does not have an effect on the calculations executed within the convolutional neural network 800 as such, since these are given solely by the structure and the weights of the edges.

A convolutional layer 811 is a connection layer between an anterior node layer 810 (with node values x(n−1)) and a posterior node layer 812 (with node values x(n)). In particular, a convolutional layer 811 is characterized by the structure and the weights of the incoming edges forming a convolution operation based on a certain number of kernels. In particular, the structure and the weights of the edges of the convolutional layer 811 are chosen such that the values x(n) of the nodes 822 of the posterior node layer 812 are calculated as a convolution x(n)=K*x(n−1) based on the values x(n−1) of the nodes 820 anterior node layer 810, where the convolution * is defined in the two-dimensional case as

$${x}_{k}^{( n )} [ i , j ] = ( K * {x}^{( n - 1 )} ) [ i , j ] = {\sum }_{{1}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 820, 822 (e.g., a 3×3 matrix, or a 5×5 matrix). In particular, this implies that the weights of the edges in the convolution layer 811 are not independent, but chosen such that they produce said convolution equation. In particular, for a kernel being a 3×3 matrix, there are only 9 independent weights (each entry of the kernel matrix corresponding to one independent weight), irrespectively of the number of nodes 820, 822 in the anterior node layer 810 and the posterior node layer 812.

In general, convolutional neural networks 800 use node layers 810, 812, 814 with a plurality of channels, in particular, due to the use of a plurality of kernels in convolutional layers 811. In those cases, the node layers can be considered as (d+1)-dimensional matrices (the first dimension indexing the channels). The action of a convolutional layer 811 is then a two-dimensional example defined as

$${x}^{{( n )}_{b}} [ i , j ] = {\sum }_{a} {K}_{a , {b}^{*}} {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 810, x⁽ⁿ⁾_{b} corresponds to the b-th channel of the posterior node layer 812 and K_{a,b} corresponds to one of the kernels. If a convolutional layer 811 acts on an anterior node layer 810 with A channels and outputs a posterior node layer 812 with B channels, there are A·B independent d-dimensional kernels K_{a,b}.

In general, in convolutional neural networks 800 activation functions are used. In this embodiment ReLU (acronym for “Rectified Linear Units”) is used, with R(z)=max(0, z), so that the action of the convolutional layer 811 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 810 comprises 36 nodes 820, arranged as a two-dimensional 6×6 matrix. The first hidden node layer 812 comprises 72 nodes 822, arranged as two two-dimensional 6×6 matrices, each of the two matrices being the result of a convolution of the values of the input layer with a 3×3 kernel within the convolutional layer 811. Equivalently, the nodes 822 of the first hidden node layer 812 can be interpreted as arranged as a three-dimensional 2×6×6 matrix, wherein the first dimension correspond to the channel dimension.

The advantage of using convolutional layers 811 is that spatially local correlation of the input data can exploited by enforcing a local connectivity pattern between nodes of adjacent layers, in particular by each node being connected to only a small region of the nodes of the preceding layer.

A pooling layer 813 is a connection layer between an anterior node layer 812 (with node values x(n−1)) and a posterior node layer 814 (with node values x(n)). In particular, a pooling layer 813 can be characterized by the structure and the weights of the edges and the activation function forming a pooling operation based on a non-linear pooling function f. For example, in the two-dimensional case the values x(n) of the nodes 824 of the posterior node layer 814 can be calculated based on the values x(n−1) of the nodes 822 of the anterior node layer 812 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ]$$

In other words, by using a pooling layer 813 the number of nodes 822, 824 can be reduced, by re-placing a number d1·d2 of neighboring nodes 822 in the anterior node layer 812 with a single node 822 in the posterior node layer 814 being calculated as a function of the values of said number of neighboring nodes. In particular, the pooling function f can be the max-function, the average or the L2-Norm. In particular, for a pooling layer 813 the weights of the incoming edges are fixed and are not modified by training.

The advantage of using a pooling layer 813 is that the number of nodes 822, 824 and the number of parameters is reduced. This leads to the amount of computation in the network being reduced and to a control of overfitting.

In the displayed embodiment, the pooling layer 813 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 800 are fully connected layers 815. A fully connected layer 815 is a connection layer between an anterior node layer 814 and a posterior node layer 816. A fully connected layer 813 can be characterized by the fact that a majority, in particular, all edges between nodes 814 of the anterior node layer 814 and the nodes 816 of the posterior node layer are present, and wherein the weight of each of these edges can be adjusted individually.

In this embodiment, the nodes 824 of the anterior node layer 814 of the fully connected layer 815 are displayed both as two-dimensional matrices, and additionally as non-related nodes (indicated as a line of nodes, wherein the number of nodes was reduced for a better presentability). This operation is also denoted as “flattening”. In this embodiment, the number of nodes 826 in the posterior node layer 816 of the fully connected layer 815 smaller than the number of nodes 824 in the anterior node layer 814. Alternatively, the number of nodes 826 can be equal or larger.

Furthermore, in this embodiment the Softmax activation function is used within the fully connected layer 815. By applying the Softmax function, the sum the values of all nodes 826 of the output layer 816 is 1, and all values of all nodes 826 of the output layer 816 are real numbers between 0 and 1. In particular, if using the convolutional neural network 800 for categorizing input data, the values of the output layer 816 can be interpreted as the probability of the input data falling into one of the different categories.

In particular, convolutional neural networks 800 can be trained based on the backpropagation algorithm. For preventing overfitting, methods of regularization can be used, e.g., dropout of nodes 820, . . . , 824, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

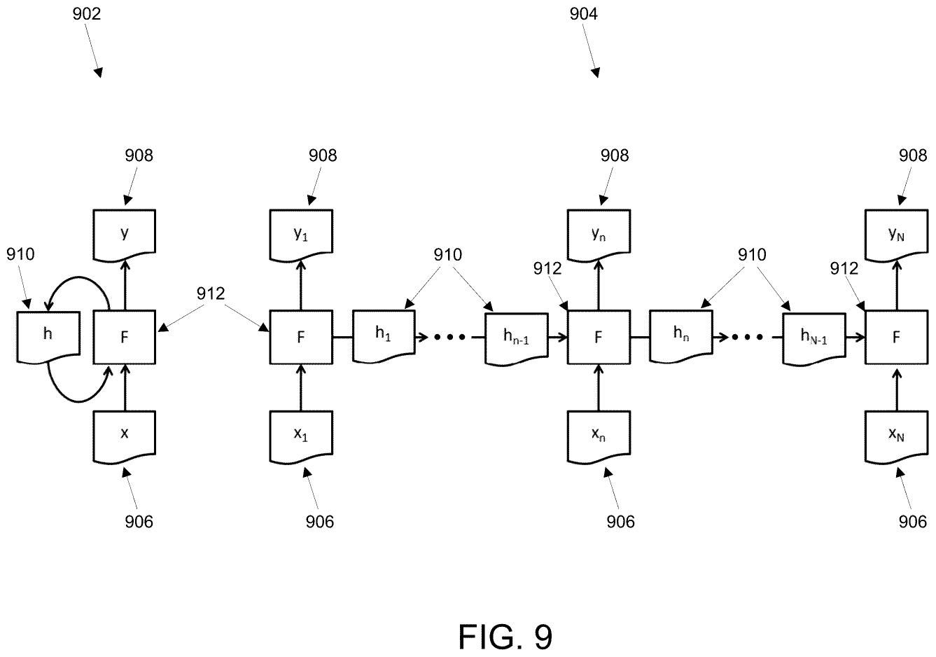

FIG. 9 shows the schematic structure of a recurrent machine learning model F, both in a recurrent representation 902 and in an unfolded representation 904, that may be used to implement one or more machine learning models described herein. The recurrent machine learning model takes as input several input datasets x, x₁, . . . , x_{N} 906 and creates a corresponding set of output datasets y, y₁, . . . , y_{N} 908. Furthermore, the output depends on a so-called hidden vector h, h₁, . . . , h_{N} 910, which implicitly comprises information about input datasets previously used as input for the recurrent machine learning model F 912. By using these hidden vectors h, h₁, . . . , h_{N} 910, a sequentiality of the input datasets can be leveraged.

In a single step of the processing, the recurrent machine learning model F 912 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 912 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 912 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1-3 or 6. Certain steps or functions of the methods and workflows described herein, including one or more of the steps or functions of FIG. 1-3 or 6, may be performed by a server or by another processor in a network-based cloud-computing system. Certain steps or functions of the methods and workflows described herein, including one or more of the steps of FIG. 1-3 or 6, may be performed by a client computer in a network-based cloud computing system. The steps or functions of the methods and workflows described herein, including one or more of the steps of FIG. 1-3 or 6, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1-3 or 6, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1002 that may be used to implement systems, apparatuses, and methods described herein is depicted in FIG. 10. Computer 1002 includes a processor 1004 operatively coupled to a data storage device 1012 and a memory 1010. Processor 1004 controls the overall operation of computer 1002 by executing computer program instructions that define such operations. The computer program instructions may be stored in data storage device 1012, or other computer readable medium, and loaded into memory 1010 when execution of the computer program instructions is desired. Thus, the method and workflow steps or functions of FIG. 1-3 or 6 can be defined by the computer program instructions stored in memory 1010 and/or data storage device 1012 and controlled by processor 1004 executing the computer program instructions. For example, the computer program instructions can be implemented as computer executable code programmed by one skilled in the art to perform the method and workflow steps or functions of FIG. 1-3 or 6. Accordingly, by executing the computer program instructions, the processor 1004 executes the method and workflow steps or functions of FIG. 1-3 or 6. Computer 1002 may also include one or more network interfaces 1006 for communicating with other devices via a network. Computer 1002 may also include one or more input/output devices 1008 that enable user interaction with computer 1002 (e.g., display, keyboard, mouse, speakers, buttons, etc.).

Processor 1004 may include both general and special purpose microprocessors, and may be the sole processor or one of multiple processors of computer 1002. Processor 1004 may include one or more central processing units (CPUs), for example. Processor 1004, data storage device 1012, and/or memory 1010 may include, be supplemented by, or incorporated in, one or more application-specific integrated circuits (ASICs) and/or one or more field programmable gate arrays (FPGAs).

Data storage device 1012 and memory 1010 each include a tangible non-transitory computer readable storage medium. Data storage device 1012, and memory 1010, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1008 may include peripherals, such as a printer, scanner, display screen, etc. For example, input/output devices 1008 may include a display device such as a cathode ray tube (CRT) or liquid crystal display (LCD) monitor for displaying information to the user, a keyboard, and a pointing device such as a mouse or a trackball by which the user can provide input to computer 1002.

An image acquisition device 1014 can be connected to the computer 1002 to input image data (e.g., medical images) to the computer 1002. It is possible to implement the image acquisition device 1014 and the computer 1002 as one device. It is also possible that the image acquisition device 1014 and the computer 1002 communicate wirelessly through a network. In a possible embodiment, the computer 1002 can be located remotely with respect to the image acquisition device 1014.

Any or all of the systems, apparatuses, and methods discussed herein may be implemented using one or more computers such as computer 1002.

One skilled in the art will recognize that an implementation of an actual computer or computer system may have other structures and may contain other components as well, and that FIG. 10 is a high-level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving one or more input medical images; performing a medical imaging analysis task based on the one or more input medical images using a fine-tuned medical foundational model; and outputting results of the medical imaging analysis task, wherein the medical foundational model is fine-tuned by: receiving weakly labeled training medical images; determining an uncertainty associated with each of the weakly labeled training medical images; selecting unconfident ones of the weakly labeled training medical images based on the uncertainty; receiving labels for the selected unconfident weakly labeled training medical images from one or more users; fine-tuning a machine learning based student network based on the received labels and the selected unconfident weakly labeled training medical images using a machine learning based teacher network, wherein the machine learning based teacher network and the machine learning based student network are initialized as being a pretrained medical foundational model during a first iteration; updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and outputting the fine-tuned machine learning based student network as the fine-tuned medical foundational model.

Illustrative embodiment 2. The computer-implemented method of illustrative embodiment 1, wherein determining an uncertainty associated with each of the weakly labeled training medical images comprises: for each respective one of the weakly labeled training medical images: applying a plurality of perturbations to the respective weakly labeled training medical image; generating a prediction for each of the plurality of perturbations using the machine learning based teacher network; and determining the uncertainty of the respective weakly labeled training medical image based on the predictions.

Illustrative embodiment 3. The computer-implemented method of any one of illustrative embodiments 1-2, wherein selecting unconfident ones of the weakly labeled training medical images based on the uncertainty comprises: comparing the uncertainty to a threshold.

Illustrative embodiment 4. The computer-implemented method of any one of illustrative embodiments 1-3, wherein selecting unconfident ones of the weakly labeled training medical images based on the uncertainty comprises: sampling the unconfident ones of the weakly labeled training medical images within ranges of the uncertainty.

Illustrative embodiment 5. The computer-implemented method of any one of illustrative embodiments 1-4, wherein: selecting unconfident ones of the weakly labeled training medical images based on the uncertainty comprises: determining confident ones of the weakly labeled training medical images based on the uncertainty; and fine-tuning a machine learning based student network based on the received labels and the selected unconfident weakly labeled training medical images using a machine learning based teacher network comprises: fine-tuning the machine learning based student network further based on the confident weakly labeled training medical images and their weak labels.

Illustrative embodiment 6. The computer-implemented method of any one of illustrative embodiments 1-5, wherein updating the machine learning based teacher network based on weights of the fine-tuned machine learning based student network comprises: updating weights of the machine learning based teacher network based on an exponential moving average of weights of the fine-tuned machine learning based student network.

Illustrative embodiment 7. The computer-implemented method of any one of illustrative embodiments 1-6, wherein fine-tuning a machine learning based student network based on the received labels and the selected unconfident weakly labeled training medical images using a machine learning based teacher network comprises: fine-tuning the machine learning based student network based on a consistency loss enforcing a consistency between outputs of the machine learning based teacher network and the machine learning based student network and a supervision loss measuring an error between the outputs of the machine learning based student network and the received labels.

Illustrative embodiment 8. An apparatus comprising: means for receiving one or more input medical images; means for performing a medical imaging analysis task based on the one or more input medical images using a fine-tuned medical foundational model; and means for outputting results of the medical imaging analysis task, wherein the medical foundational model is fine-tuned by: receiving weakly labeled training medical images; determining an uncertainty associated with each of the weakly labeled training medical images; selecting unconfident ones of the weakly labeled training medical images based on the uncertainty; receiving labels for the selected unconfident weakly labeled training medical images from one or more users; fine-tuning a machine learning based student network based on the received labels and the selected unconfident weakly labeled training medical images using a machine learning based teacher network, wherein the machine learning based teacher network and the machine learning based student network are initialized as being a pretrained medical foundational model during a first iteration; updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and outputting the fine-tuned machine learning based student network as the fine-tuned medical foundational model.

Illustrative embodiment 9. The apparatus of illustrative embodiment 8, wherein determining an uncertainty associated with each of the weakly labeled training medical images comprises: for each respective one of the weakly labeled training medical images: applying a plurality of perturbations to the respective weakly labeled training medical image; generating a prediction for each of the plurality of perturbations using the machine learning based teacher network; and determining the uncertainty of the respective weakly labeled training medical image based on the predictions.

Illustrative embodiment 10. The apparatus of any one of illustrative embodiments 8-9, wherein selecting unconfident ones of the weakly labeled training medical images based on the uncertainty comprises: comparing the uncertainty to a threshold.

Illustrative embodiment 11. The apparatus of any one of illustrative embodiments 8-10, wherein selecting unconfident ones of the weakly labeled training medical images based on the uncertainty comprises: sampling the unconfident ones of the weakly labeled training medical images within ranges of the uncertainty.

Illustrative embodiment 12. A non-transitory computer-readable storage medium comprising instructions which, when executed by a computer, cause the computer to carry out operations comprising: receiving one or more input medical images; performing a medical imaging analysis task based on the one or more input medical images using a fine-tuned medical foundational model; and outputting results of the medical imaging analysis task, wherein the medical foundational model is fine-tuned by: receiving weakly labeled training medical images; determining an uncertainty associated with each of the weakly labeled training medical images; selecting unconfident ones of the weakly labeled training medical images based on the uncertainty; receiving labels for the selected unconfident weakly labeled training medical images from one or more users; fine-tuning a machine learning based student network based on the received labels and the selected unconfident weakly labeled training medical images using a machine learning based teacher network, wherein the machine learning based teacher network and the machine learning based student network are initialized as being a pretrained medical foundational model during a first iteration; updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and outputting the fine-tuned machine learning based student network as the fine-tuned medical foundational model.

Illustrative embodiment 13. The non-transitory computer-readable storage medium of illustrative embodiment 12, wherein: selecting unconfident ones of the weakly labeled training medical images based on the uncertainty comprises: determining confident ones of the weakly labeled training medical images based on the uncertainty; and fine-tuning a machine learning based student network based on the received labels and the selected unconfident weakly labeled training medical images using a machine learning based teacher network comprises: fine-tuning the machine learning based student network further based on the confident weakly labeled training medical images and their weak labels.

Illustrative embodiment 14. The non-transitory computer-readable storage medium of any one of illustrative embodiment 12-13, wherein updating the machine learning based teacher network based on weights of the fine-tuned machine learning based student network comprises: updating weights of the machine learning based teacher network based on an exponential moving average of weights of the fine-tuned machine learning based student network.

Illustrative embodiment 15. The non-transitory computer-readable storage medium of any one of illustrative embodiment 12-14, wherein fine-tuning a machine learning based student network based on the received labels and the selected unconfident weakly labeled training medical images using a machine learning based teacher network comprises: fine-tuning the machine learning based student network based on a consistency loss enforcing a consistency between outputs of the machine learning based teacher network and the machine learning based student network and a supervision loss measuring an error between the outputs of the machine learning based student network and the received labels.

Illustrative embodiment 16. A computer-implemented method for fine-tuning a pretrained medical foundation model, comprising: receiving weakly labeled training medical images; determining an uncertainty associated with each of the weakly labeled training medical images; selecting unconfident ones of the weakly labeled training medical images based on the uncertainty; receiving labels for the selected unconfident weakly labeled training medical images from one or more users; fine-tuning a machine learning based student network based on the received labels and the selected unconfident weakly labeled training medical images using a machine learning based teacher network, wherein the machine learning based teacher network and the machine learning based student network are initialized as being the pretrained medical foundational model during a first iteration; updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and outputting the fine-tuned machine learning based student network as a fine-tuned medical foundational model.

Illustrative embodiment 17. The computer-implemented method of illustrative embodiment 16, wherein determining an uncertainty associated with each of the weakly labeled training medical images comprises: for each respective one of the weakly labeled training medical images: applying a plurality of perturbations to the respective weakly labeled training medical image; generating a prediction for each of the plurality of perturbations using the machine learning based teacher network; and determining the uncertainty of the respective weakly labeled training medical image based on the predictions.

Illustrative embodiment 18. The computer-implemented method of any one of illustrative embodiment 16-17, wherein selecting unconfident ones of the weakly labeled training medical images based on the uncertainty comprises: comparing the uncertainty to a threshold.

Illustrative embodiment 19. The computer-implemented method of any one of illustrative embodiment 16-18, wherein selecting unconfident ones of the weakly labeled training medical images based on the uncertainty comprises: sampling the unconfident ones of the weakly labeled training medical images within ranges of the uncertainty.

Illustrative embodiment 20. The computer-implemented method of any one of illustrative embodiment 16-19, wherein: selecting unconfident ones of the weakly labeled training medical images based on the uncertainty comprises: determining confident ones of the weakly labeled training medical images based on the uncertainty; and fine-tuning a machine learning based student network based on the received labels and the selected unconfident weakly labeled training medical images using a machine learning based teacher network comprises: fine-tuning the machine learning based student network further based on the confident weakly labeled training medical images and their weak labels.

1. A computer-implemented method comprising:
receiving one or more input medical images;
performing a medical imaging analysis task based on the one or more input medical images using a fine-tuned medical foundational model; and
outputting results of the medical imaging analysis task,
wherein the medical foundational model is fine-tuned by:
receiving weakly labeled training medical images;
determining an uncertainty associated with each of the weakly labeled training medical images;
selecting unconfident ones of the weakly labeled training medical images based on the uncertainty;
receiving labels for the selected unconfident weakly labeled training medical images from one or more users;
fine-tuning a machine learning based student network based on the received labels and the selected unconfident weakly labeled training medical images using a machine learning based teacher network, wherein the machine learning based teacher network and the machine learning based student network are initialized as being a pretrained medical foundational model during a first iteration;
updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and
outputting the fine-tuned machine learning based student network as the fine-tuned medical foundational model.

2. The computer-implemented method of claim 1, wherein determining an uncertainty associated with each of the weakly labeled training medical images comprises:
for each respective one of the weakly labeled training medical images:
applying a plurality of perturbations to the respective weakly labeled training medical image;
generating a prediction for each of the plurality of perturbations using the machine learning based teacher network; and
determining the uncertainty of the respective weakly labeled training medical image based on the predictions.

3. The computer-implemented method of claim 1, wherein selecting unconfident ones of the weakly labeled training medical images based on the uncertainty comprises:
comparing the uncertainty to a threshold.

4. The computer-implemented method of claim 1, wherein selecting unconfident ones of the weakly labeled training medical images based on the uncertainty comprises:
sampling the unconfident ones of the weakly labeled training medical images within ranges of the uncertainty.

5. The computer-implemented method of claim 1, wherein:
selecting unconfident ones of the weakly labeled training medical images based on the uncertainty comprises:
determining confident ones of the weakly labeled training medical images based on the uncertainty; and
fine-tuning a machine learning based student network based on the received labels and the selected unconfident weakly labeled training medical images using a machine learning based teacher network comprises:
fine-tuning the machine learning based student network further based on the confident weakly labeled training medical images and their weak labels.

6. The computer-implemented method of claim 1, wherein updating the machine learning based teacher network based on weights of the fine-tuned machine learning based student network comprises:
updating weights of the machine learning based teacher network based on an exponential moving average of weights of the fine-tuned machine learning based student network.

7. The computer-implemented method of claim 1, wherein fine-tuning a machine learning based student network based on the received labels and the selected unconfident weakly labeled training medical images using a machine learning based teacher network comprises:
fine-tuning the machine learning based student network based on a consistency loss enforcing a consistency between outputs of the machine learning based teacher network and the machine learning based student network and a supervision loss measuring an error between the outputs of the machine learning based student network and the received labels.

8. An apparatus comprising:
means for receiving one or more input medical images;
means for performing a medical imaging analysis task based on the one or more input medical images using a fine-tuned medical foundational model; and
means for outputting results of the medical imaging analysis task,
wherein the medical foundational model is fine-tuned by:
receiving weakly labeled training medical images;
determining an uncertainty associated with each of the weakly labeled training medical images;
selecting unconfident ones of the weakly labeled training medical images based on the uncertainty;
receiving labels for the selected unconfident weakly labeled training medical images from one or more users;
fine-tuning a machine learning based student network based on the received labels and the selected unconfident weakly labeled training medical images using a machine learning based teacher network, wherein the machine learning based teacher network and the machine learning based student network are initialized as being a pretrained medical foundational model during a first iteration;
updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and
outputting the fine-tuned machine learning based student network as the fine-tuned medical foundational model.

9. The apparatus of claim 8, wherein determining an uncertainty associated with each of the weakly labeled training medical images comprises:
for each respective one of the weakly labeled training medical images:
applying a plurality of perturbations to the respective weakly labeled training medical image;
generating a prediction for each of the plurality of perturbations using the machine learning based teacher network; and
determining the uncertainty of the respective weakly labeled training medical image based on the predictions.

10. The apparatus of claim 8, wherein selecting unconfident ones of the weakly labeled training medical images based on the uncertainty comprises:
comparing the uncertainty to a threshold.

11. The apparatus of claim 8, wherein selecting unconfident ones of the weakly labeled training medical images based on the uncertainty comprises:
sampling the unconfident ones of the weakly labeled training medical images within ranges of the uncertainty.

12. A non-transitory computer-readable storage medium comprising instructions which, when executed by a computer, cause the computer to carry out operations comprising:
receiving one or more input medical images;
performing a medical imaging analysis task based on the one or more input medical images using a fine-tuned medical foundational model; and
outputting results of the medical imaging analysis task,
wherein the medical foundational model is fine-tuned by:
receiving weakly labeled training medical images;
determining an uncertainty associated with each of the weakly labeled training medical images;
selecting unconfident ones of the weakly labeled training medical images based on the uncertainty;
receiving labels for the selected unconfident weakly labeled training medical images from one or more users;
fine-tuning a machine learning based student network based on the received labels and the selected unconfident weakly labeled training medical images using a machine learning based teacher network, wherein the machine learning based teacher network and the machine learning based student network are initialized as being a pretrained medical foundational model during a first iteration;
updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and
outputting the fine-tuned machine learning based student network as the fine-tuned medical foundational model.

13. The non-transitory computer-readable storage medium of claim 12, wherein:
selecting unconfident ones of the weakly labeled training medical images based on the uncertainty comprises:
determining confident ones of the weakly labeled training medical images based on the uncertainty; and
fine-tuning a machine learning based student network based on the received labels and the selected unconfident weakly labeled training medical images using a machine learning based teacher network comprises:
fine-tuning the machine learning based student network further based on the confident weakly labeled training medical images and their weak labels.

14. The non-transitory computer-readable storage medium of claim 12, wherein updating the machine learning based teacher network based on weights of the fine-tuned machine learning based student network comprises:
updating weights of the machine learning based teacher network based on an exponential moving average of weights of the fine-tuned machine learning based student network.

15. The non-transitory computer-readable storage medium of claim 12, wherein fine-tuning a machine learning based student network based on the received labels and the selected unconfident weakly labeled training medical images using a machine learning based teacher network comprises:
fine-tuning the machine learning based student network based on a consistency loss enforcing a consistency between outputs of the machine learning based teacher network and the machine learning based student network and a supervision loss measuring an error between the outputs of the machine learning based student network and the received labels.

16. A computer-implemented method for fine-tuning a pretrained medical foundation model, comprising:
receiving weakly labeled training medical images;
determining an uncertainty associated with each of the weakly labeled training medical images;
selecting unconfident ones of the weakly labeled training medical images based on the uncertainty;
receiving labels for the selected unconfident weakly labeled training medical images from one or more users;
fine-tuning a machine learning based student network based on the received labels and the selected unconfident weakly labeled training medical images using a machine learning based teacher network, wherein the machine learning based teacher network and the machine learning based student network are initialized as being the pretrained medical foundational model during a first iteration;
updating the machine learning based teacher network based on weights of the fine-tuned machine learning based student network and repeating the determining, the selecting, the receiving, and the fine-tuning for one or more additional iterations, wherein the machine learning based student network is initialized as being the updated machine learning based teacher network during the one or more additional iterations; and
outputting the fine-tuned machine learning based student network as a fine-tuned medical foundational model.

17. The computer-implemented method of claim 16, wherein determining an uncertainty associated with each of the weakly labeled training medical images comprises:
for each respective one of the weakly labeled training medical images:
applying a plurality of perturbations to the respective weakly labeled training medical image;
generating a prediction for each of the plurality of perturbations using the machine learning based teacher network; and
determining the uncertainty of the respective weakly labeled training medical image based on the predictions.

18. The computer-implemented method of claim 16, wherein selecting unconfident ones of the weakly labeled training medical images based on the uncertainty comprises:
comparing the uncertainty to a threshold.

19. The computer-implemented method of claim 16, wherein selecting unconfident ones of the weakly labeled training medical images based on the uncertainty comprises:
sampling the unconfident ones of the weakly labeled training medical images within ranges of the uncertainty.

20. The computer-implemented method of claim 16, wherein:
selecting unconfident ones of the weakly labeled training medical images based on the uncertainty comprises:
determining confident ones of the weakly labeled training medical images based on the uncertainty; and
fine-tuning a machine learning based student network based on the received labels and the selected unconfident weakly labeled training medical images using a machine learning based teacher network comprises:
fine-tuning the machine learning based student network further based on the confident weakly labeled training medical images and their weak labels.
